(19) United States
(12) Patent Application Publication
(10) Pub. No.: US 2018/0236232 A1
(43) Pub. Date: Aug. 23, 2018

(54) METHODS AND SYSTEMS FOR ACOUSTIC STIMULATION OF BRAIN WAVES

(71) Applicant: RYTHM, Paris (FR)

(72) Inventors: Quentin Soulet De Brugiere, Pyla Sur Mer (FR); Hugo Mercier, Chilly Mazarin (FR); Valentin Thorey, Paris (FR); Oliver Tranzer, Paris (FR)

(73) Assignee: RYTHM, Paris (FR)

(21) Appl. No.: 15/750,288

(22) PCT Filed: Aug. 4, 2016
(86) PCT No.: PCT/FR2016/052031

§ 371 Date: Feb. 5, 2018

(30) Foreign Application Priority Data
Aug. 4, 2015 (FR) 15 57523

(51) Int. Cl. A61N 1/36; A61B 5/04; A61B 5/00; A61B 5/0482; A61B 5/048

(52) U.S. Cl. CPC A61N 1/36031; A61B 5/04001; A61B 5/6803; A61N 1/0408; A61B 5/048; A61B 5/4809; A61N 1/36034; A61B 5/0482

(57) ABSTRACT

A method and system for personalized acoustic brain wave stimulation of a person. The system comprises an acoustic stimulation device and a remote server. The device comprises a memory able to store operating parameters, acquisition element of a measured signal analysis element in order to assess whether the person is in a state susceptible to stimulation and emission element designed for emitting an acoustic signal. The acquisition element, analysis element and/or emission element operate depending on operating parameters. The device and the remote server comprise means of data transmission for transmitting operating data from the device to the server and transmitting second operating parameters from the server to the device. The remote server comprises means of processing operating data for determining second operating parameters.

### FIELD OF THE INVENTION

The present invention relates to methods and systems for personalized acoustic brain wave stimulation of a person.

### BACKGROUND OF THE INVENTION

Methods are known with which to stimulate a person's brain waves, in particular during different sleep phases.

Thus, for example, document WO 2008/039930 describes an example of such a method in which brain wave stimulation is implemented in order to promote the generation of slow brain waves during deep sleep.

During such a method, a power spectrum of a person's encephalogram is analyzed for determining the sleep stage reached by said person. When the person is considered to have reached a deep sleep stage, a periodic stimulation is emitted at a predefined frequency for a preset time. The stimulation frequency is defined ahead for being close to a frequency of slow brain waves.

Just the same, such a method has disadvantages. In fact, each person has a specific brain activity which additionally changes over time with the person's physical and mental condition. Such a stimulation method works more or less well depending on the person to whom it is applied and, for just one person, depending on the time at which it is applied.

The purpose of the present invention is especially to improve this situation.

For this purpose, the subject of the invention is a method for personalized acoustic brain wave stimulation of a person comprising:

A first step of acoustic brain wave stimulation of a person implemented by a device for acoustic brain wave stimulation which the person could wear, wherein the stimulation device comprises memory storing first operating parameters, and the initial stimulation step comprises the substeps:

a) Acquisition of at least one measured signal representative of a physiological signal from the person;

b) Analysis of the measured signal in order to assess whether the person is in a state susceptible to stimulation; and

c) If the assessment shows that the person is in a state susceptible to stimulation, emitting an acoustic signal, audible by the person, and synchronized with the person's predefined brain wave temporal pattern,

Wherein at least one of the substeps of acquisition a), analysis b), and emission c) is done depending on the first operating parameters;
A step of sending operating data from the device to a remote server, wherein said operating data comprise said measured signal;
A step of operating data processing, on the remote server, for determining second operating parameters;

A1 step of transmission and storage of the second operating parameters from the remote server into the memory of the device; and

A second step of acoustic brain wave stimulation implemented by the device in which the memory of the device stores the second operating parameters, and in which at least one of the substeps of acquisition a), analysis b), and emission c) is done depending on the second operating parameters.

In preferred embodiments of the invention, use can further be made of one and/or another of the following dispositions:
The operating parameters comprise at least one parasitic frequency of the measured signal,

And the substep of acquisition a) comprises a frequency filtering of said parasitic frequency in the measured signal;
The operating parameters comprise at least one energy threshold for a measured signal spectrum,

And the substep of analysis b) comprises the comparison of an energy from a measured signal spectrum with said threshold;
The operating parameters comprise at least one temporal frequency threshold for a predefined pattern from the measured signal,

And the substep of analysis b) comprises the identification of said predefined pattern in the measured signal and the comparison of a frequency of said pattern in the measured signal with said threshold;
The operating parameters comprise at least one muscular activity level threshold,

And the substep of analysis b) comprises the determination of a muscular activity level from the measured signal and the comparison of said muscular activity level with said threshold;
The substep of analysis b) is implemented at least in part by an algorithm for automatic classification of measured data determined from the measured signal,

And the operating parameters comprise at least one parameter from said automatic classification algorithm and/or one database of classes from said automatic classification algorithm;
The operating parameters comprise at least one parameter selected from a list including sound level, length, spectrum and time pattern of an acoustic signal,

And the acoustic signal emitted during the substep of emission c) is emitted based on said parameters;
The operating parameters comprise at least one parameter selected from a list including a person's brain wave phase and a predefined brain wave temporal pattern of the person,

And the acoustic signal emitted during the substep of emission c) is emitted so as to be synchronized based on said parameters;
The processing step comprises analyzing the measured signal on the remote server for identifying at least one parasitic frequency of the measured signal,

And the second operating parameters are determined depending on said parasitic frequency;
The processing step comprises searching the measured signal for at least one predefined pattern indicating wakening or beginning of wakening of the person and following emitting an acoustic signal, so as to determine an indicator of wakening under the effect of the stimulation,

And the second operating parameters are determined depending on said wakening indicator;
The processing step comprises comparing a portion of the measured signal acquired after emitting an acoustic signal with a baseline portion of the measured signal, so as to determine a stimulation impact indicator,

And the second operating parameters are determined depending on said impact indicator;
Determining the second operating parameters depending on the stimulation impact indicator includes implementing an automatic classification algorithm, wherein said automatic classification algorithm is preferably defined during a preliminary automatic learning step;
The first step of acoustic brain wave stimulation of a person is implemented a plurality of times by a plurality of respective devices, respectively suited to be worn by a plurality of respective people,

The step of transmitting operating data to the remote server is implemented a plurality of times from said plurality of respective devices, so as to respectively send a plurality of respective operating data, respectively comprising at least one measured signal from each of said respective devices,

And the processing step comprises the analysis of said plurality of operating data so as to determine second operating parameters to be sent and stored in the memory of at least one device among the plurality of devices;
The first stimulation step is repeated a plurality of times by a device during a time of operating said device,

And the step of transmitting operating data to a remote server from the device is implemented after said operating time, wherein the operating data comprise at least one measured signal for each of the repetitions of the first stimulation step;
The operating time of the device extends over several hours, preferably at least eight hours;
Emitting an acoustic signal synchronized with a predefined brain wave temporal pattern comprises:

Determining a brain wave temporal shape from the measured signal,

Determining from said brain wave temporal shape at least one instant for synchronization of the predefined brain wave temporal pattern with a predefined acoustic signal temporal pattern, and

Commanding an acoustic transducer of the device so that the predefined acoustic signal temporal pattern is sent at said synchronization instant;
The brain wave is a slow brain wave having a frequency below 5 Hz and over 0.3 Hz;
The acoustic signal is an intermittent signal and an acoustic signal length is less than a brain wave period, preferably less than a few seconds, preferably less than one second;
The acoustic signal is a continuous signal and an acoustic signal length is greater than a brain wave period;
The predefined brain wave temporal pattern corresponds to a brain wave local temporal maximum, a brain wave local temporal minimum, a rising or descending front of a brain wave local maximum or minimum, a predefined succession or at least one brain wave local temporal maximum or minimum, or a rising or descending front of such a succession.

The subject of the invention is also a personalized acoustic brain wave stimulation system for a person, wherein the system comprises an acoustic brain wave stimulation device for a person and a remote server,

wherein the device comprises:
A memory adapted to to store operating parameters comprising at least one among first operating parameters and second operating parameters;
Means of acquisition at least one measured signal representative of a physiological signal from the person;
Means of analysis of the measured signal in order to assess whether the person is in a state susceptible to stimulation; and
Means of emission designed for emitting an acoustic signal, audible by the person, and synchronized with the person's predefined brain wave temporal pattern if it is assessed that the person is in a state susceptible to stimulation;

Wherein the at least one among the means of acquisition, means of analysis and means of emission can operate depending on operating parameters stored in memory;

Wherein the device and the remote server comprise respective means of data transmission designed for:
Transmitting operating data from the device to the remote server, wherein said operating data comprises at least said measured signal; and
Transmitting second operating parameters from the remote server to the device and storing said second operating parameters in the memory of the device;

And wherein the remote server comprises means of processing operating data in order to determine second operating parameters.

Other features and advantages of the invention will become apparent during the following description of several embodiments thereof, given as nonlimiting examples, with reference to the attached drawings.

### IN THE DRAWINGS

FIG. 1 is a schematic view of device for acoustic brain wave stimulation of a person according to an embodiment of the invention;

FIG. 2 is a detailed perspective view of the device from FIG. 1 wherein the device in particular comprises respectively a first and second acoustic transducer capable of emitting acoustic signals stimulating respectively a right inner ear and a left inner ear of the person;

FIG. 3 is a synoptic drawing of a system according to an embodiment of the invention comprising a device and a remote server;

FIG. 4 is a synoptic drawing of a system according to another embodiment of the invention comprising a plurality of devices and a remote server;

FIG. 5 is a flowchart illustrating an embodiment of a customization method for acoustic brain wave stimulation of a person according to an embodiment of the invention;

FIG. 6 shows a slow brain wave temporal shape, an acoustic signal and temporal patterns predefined according to an embodiment of the invention.

In the various figures, the same references designate identical or similar items.

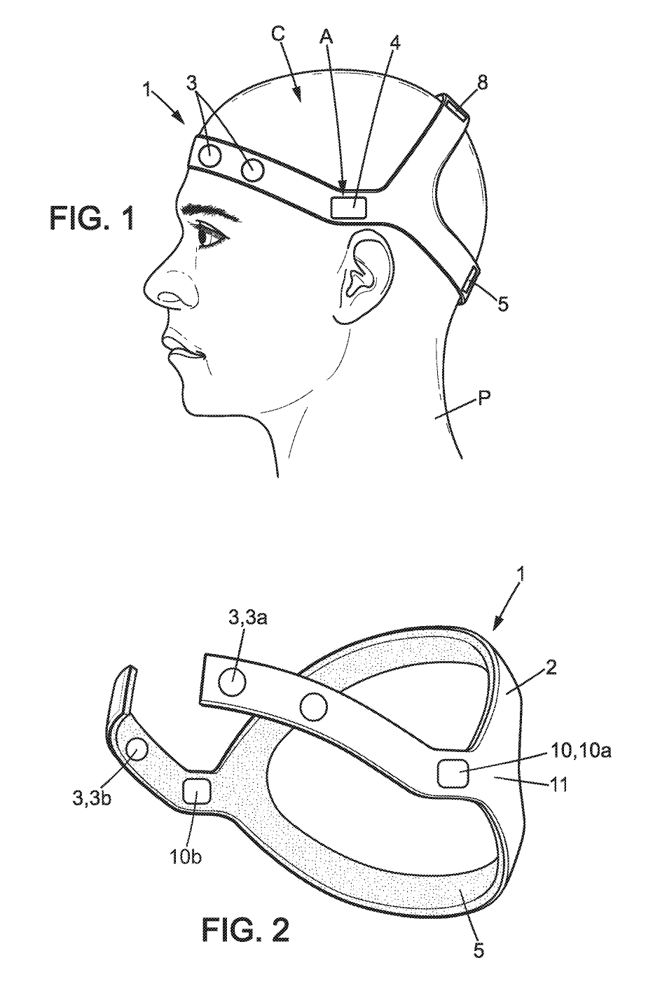

As shown in FIGS. 1 to 4, the subject of the invention is a system 1000 for personalized acoustic brain wave stimulation of a person P.

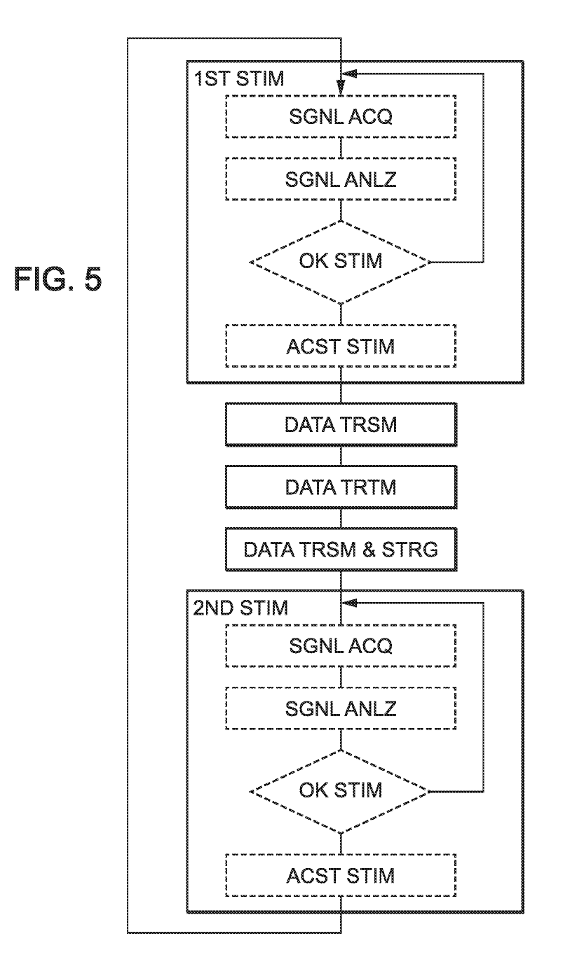

The system 1000 can implement a personalization method for acoustic brain wave stimulation of the person P who is shown in particular in FIG. 5.

The system 1000 comprises an acoustic stimulation device 1 and a remote server 10.

The device 1 can be worn by the person P, for example while the person sleeps.

For example, the device can be worn on the head of the person P.

For this purpose, the device 1 may comprise one or more support elements 2 which can at least partially surround the head of the person P so as to be retained there. The support elements 2 take, for example, the shape of one or more branches which can be disposed so as to surround the head of the person P for keeping the device 1 in place.

The device 1 can also be divided into one or more elements, which can be worn on different parts of the body of the person P, for example on the head, wrist or even the torso.

The device 1 also comprises means of acquisition 3 of at least one measured signal, means of emission 4 designed for emitting an acoustic signal audible by the person P, means of analysis 5 of the measured signal and at least one memory 6.

With the means of acquisition 3, the means of emission 4, the means of analysis and the memory 6, the device 1 is able to implement a step of acoustic brain wave stimulation of person P which is now going to be described in more detail.

This step of acoustic brain wave stimulation of the person P can be repeated one or more times.

Thus in particular, the stimulation step can be repeated a plurality of times by the device 1 during a time of operation of the device, for example while the person P sleeps.

Such a time of operation of the device can extend over several hours, for example at least eight hours, meaning about one night's sleep.

In an embodiment of the invention, the device 1 can implement the stimulation step over an operating time without communicating with the remote server 100, meaning operating independently during the operating time. In this way, the exposure of the person P to electromagnetic radiation can in particular be reduced.

Thus, for example, the device 1 can comprise a battery 8. The battery 8 can be mounted on the support element 2 as described above for the means of acquisition 3, the means of emission 4 and the means of analysis 5. The battery 8 can in particular be capable of supplying the means of acquisition 3, means of emission 4, means of analysis 5, memory 6 and communication module 7. Preferably, the battery 8 is capable of supplying energy for several hours without recharging, preferably at least eight hours so as to cover an average sleeping time of a person P.

In this way, the device 1 can operate independently over a sleeping time of the person P. In this way in particular, the device 1 is independent and capable of implementing one or more slow brain wave stimulation operations without communicating with an outside server 100, in particular without communicating with an outside server 100 over several minutes, preferably several hours, preferably at least eight hours.

“Independent” is thus understood to mean that the device can operate for an extended time, several minutes, preferably several hours, in particular at least eight hours, without needing to be recharged with electric energy, communicate with external elements such as the remote server or even to be structurally connected to an external device like an attachment element such as an arm or hanger.

In this way, the device can be used in the daily life of a person P without imposing specific constraints.

In order to implement the acoustic stimulation step, the means of acquisition 3, means of emission 4, means of analysis 5 and the memory 6 are additionally functionally connected to each other and capable of exchanging information and commands.

For this purpose, the means of acquisition 3, means of emission 4, means of analysis 5 and the memory 6 are mounted on the support element 2 so as to be so close to each other that communication between these elements 3, 4, 5, 6 is particularly quick and at high data rate.

The memory 6 can in particular be permanently mounted on the support element 2 or can be a removable module, for example a memory card such as an SD card (for “Secure Digital”).

The memory 6 is capable of recording data which will be described in the remainder of the description and can include at least one of the following elements: a measured signal S acquired by the means of acquisition 3, operating parameters of the device 1.

The operating parameters can in particular be the first operating parameters or the second operating parameters, as will be given in detail below.

In particular, the device 1 can be configured such that only one set of operating parameters stored in the memory 6 is used at a given time. For that purpose, the memory 6 can for example store operating parameters which are, mutually exclusively, either first operating parameters or second operating parameters such as defined below.

The memory 6 can be dynamically updated such that the measured signal and/or the operating parameters recorded in the memory 6 can be modified during operation of the device 1, as will be described in more detail in the remainder of the description.

The stimulation step can thus include first a substep of acquisition of the at least one measured signal S by means of the means of acquisition 3.

The measured signal S can in particular be representative of a physiological electrical signal E of the person P.

The physiological electrical signal F can for example comprise an electroencephalogram (EEG), electromyogram (EMG), electrooculogram (EOG), electrocardiogram (ECG) or any other biosignal that can be measured on the person P.

For this purpose, the means of acquisition 3 for example comprise a plurality of electrodes 3 capable of being in contact with the person P, and in particular with the skin of the person P for acquiring at least one measured signal S representative of a physiological electrical signal E of the person P.

The physiological electrical signal E advantageously comprises an electroencephalogram (EEG) of the person P.

For this purpose, in an embodiment of the invention, the device 1 comprises at least two electrodes 3 including at least one reference electrode 3a and at least one EEG measurement electrode 3b.

The device 1 can further comprise a ground electrode 3c.

In a specific embodiment, the device 1 comprises at least three EEG measurement electrodes 3c, so as to acquire physiological electrical signals E comprising at least three electroencephalogram measurement channels.

The EEG measurement electrodes 3c are arranged for example on the surface of the scalp of the person P.

In other embodiments, the device 1 can further comprise an EMG measurement electrode and, optionally, an EOG measurement electrode.

The measurement electrodes 3 can be reusable or disposable electrodes. Advantageously, the measurement electrodes 3 are reusable electrodes so as to simplify the daily use of the device.

The measurement electrodes 3 can in particular be dry electrodes or electrodes covered with a contact gel. The electrodes 3 can also be textile or silicone electrodes.

The means of acquisition 3 can also comprise measured signal S acquisition devices that are not solely electrical.

A measured signal S can thus be, generally, representative of a physiological signal of the person P.

The measured signal S can in particular be representative of a non-electrical or not totally electrical physiological signal of the person P, for example a signal of heart activity, such as heart rhythm, body temperature of the person P or even movements of the person P.

For this purpose, the means of acquisition 3 can comprise a heart rhythm detector, a body thermometer, an accelerometer, a respiration sensor, a bio-impedance sensor or even a microphone.

The means of acquisition 3 can again comprise measured signal S acquisition devices representative of the environment of the person P.

The measured signal S can thus be representative of air quality of the air surrounding the person P, for example a carbon dioxide or oxygen level, or even a temperature or ambient noise level.

Finally, the means of acquisition 3 can comprise user input devices with which the person P can enter information such as a subjective night quality index or even a subjective number of times that the person thinks they were woken up by the device 1.

The measured signal S can then be representative of information from the person P.

In an embodiment of the invention, the measured signal S acquisition substep also comprises preprocessing of the measured signal S.

Preprocessing of the measured signal S can for example comprise at least one of the following preprocessings:
Frequency filtering, for example filtering of the measured signal S by frequency and/or wavelets in a temporal frequency range of interest, for example a frequency range included in a range from 0.3 Hz to 100 Hz;
Filtering of parasitic frequencies from the measured signal S by frequency and/or by wavelets, for example capable of filtering at least one parasitic frequency from the measured signal S, for example a parasitic frequency belonging to a frequency range from 0.3 Hz to 100 Hz;
Eliminating predefined artifacts from the measured signal S.

For this purpose, one or more parasitic frequencies can be predefined and recorded in the memory 6 of the device 1. Similarly, one or more artifacts can be predefined and recorded in the memory 6 of the device 1, for example in the form of predefined patterns of the measured signal S.

Said one or more parasitic frequencies and/or the artifacts can form operating parameters of the device 1.

Said one or more parasitic frequencies and/or the artifacts can vary over time, such that the preprocessing of the signal S is variable over time.

Said one or more parasitic frequencies and/or the artifacts can in particular vary depending on absolute or relative time.

“Absolute time” is understood to be a time independent of the operation of the device, for example an hour, day of the week, month, a moment in the calendar of the person P (vacation or holiday period, biological rhythm of the person P).

“Relative time” is understood to be a time passed since an event detected by the device, for example time passed since a preceding determination of susceptibility to stimulation, time passed since the preceding stimulation or even time passed since a preceding identification of wakening or start of wakening of the person P.

Preprocessing of the measured signal S can also comprise preprocessing such as:
Amplifying, for example amplifying the measured signal S by a factor ranging from 10³ to 10⁶; and/or
Sampling the measured signal S using a digital-to-analog converter able, for example to sample the measured signal S with a sampling rate of several hundred hertz, for example 256 Hz or 512 Hz.

Such preprocessing of the measured signal S can for example be implemented by an analog or digital module of the acquisition means 3. Thus, in particular, the means of acquisition 3 can comprise active electrodes capable of performing one of the pretreatments detailed above.

The means of analysis 5 receives the measured signals S from the means of acquisition 3, which could be preprocessed as detailed above.

Based on the measured signals S, the means of analysis 5 can assess during a substep of analysis of the measured signal whether the person P is in a state susceptible to stimulation.

In a first embodiment, “a state susceptible to stimulation” is understood to mean that when the stimulation must preferably be done while sleeping, the means of analysis 5 are able to estimate whether the person P is in a state of sufficiently deep sleep in order to be able to undergo auditory stimulation without risk of being woken or that the auditory stimulation does not trigger a beginning of wakening.

In a variant or in addition, “a state susceptible to stimulation” can also mean that the means of analysis 5 are able to estimate whether the person P is in a state of sleep in which auditory stimulation could have a desired effect. Thus, the means of analysis 5 can be suited to estimate whether the person P is in a state of deep sleep such that an auditory stimulation might have an effect of lengthening the time of said deep sleep.

The means of analysis 5 are in that way for example capable of determining an index of susceptibility to stimulation from measured signals S.

Such an “index of susceptibility to stimulation” can for example be a binary index having a value “suitable for stimulation” and a value “unsuitable for stimulation”. In variants, the index of susceptibility to stimulation can take intermediate values, indicating for example a percentage of susceptibility to stimulation for the state between the extreme values indicated above.

To do that, the means of analysis 5 can analyze a heart activity signal, body temperature or even movements of the person P.

The means of analysis 5 can also analyze at least one measured signal S representative of a physiological electrical signal E of the person P.

Thus, the depth of sleep of the person P can be evaluated by an analysis of the brain, eye and muscular activity measurements.

The means of analysis 5 can for example implement one or more predefined shape recognition algorithms on the measured signal S so as to identify slow oscillations, K-complexes, spindles, an alpha rhythm, or even wakening in the measured signal S.

In a first embodiment, a frequency spectrum of the measured signal S can be determined. The predefined shapes are then determined from an energy variation of the frequency spectrum in the predefined frequency bands, such as for example an alpha (8 12 Hz), beta (>12 Hz), delta (<4Hz) or even theta wave (4 7 Hz) frequency band.

A frequency energy spectrum in one or more of said frequency bands can be calculated, for example by using a short-time Fast Fourier Transform.

In another embodiment, which could be combined with the first embodiment shown, the predefined shapes can be determined directly in the temporal shape of the measured signal F, in particular by seeking one or more predefined patterns in the measured signal S.

Thus, for example, slow oscillations and K-complexes can be detected by looking for consecutive zeros spaced less than about one second apart and by looking for a peak-to-peak maximum.

When said peak-to-peak maximum exceeds some threshold, a slow wave or a K-complex can then be recorded.

The means of analysis 5 can also estimate whether the person P is in a state susceptible to stimulation from a measured signal representative of eye-movement, for example an electrooculogram.

For this purpose, the means of analysis 5 can for example calculate a sliding average of a variation of the eye movement.

The means of analysis 5 can again estimate whether the person P is in a state susceptible to stimulation from a measured signal representative of a muscular activity level.

For implementing the analysis substep, the means of analysis 5 can then compare each of said magnitudes calculated from the measured signal with a predefined threshold for estimating whether the person P is in a state susceptible to stimulation, for example sufficiently asleep for receiving stimulation.

The result of this comparison can provide an index of susceptibility to stimulation such as defined above.

Thus, for example, the means of analysis 5 can compare an energy spectrum of the measured signal S with a predefined threshold for energy spectrum of the measured signal S.

The means of analysis 5 can also compare a frequency of a predefined pattern identified in the measured signal with a predefined temporal frequency threshold of said pattern in the measured signal.

The means of analysis 5 can again compare a muscular activity level with a predefined threshold for muscular activity level.

In this way, a plurality of thresholds can be predefined and recorded in the memory 6 of the device 1 and form operating parameters of the device 1.

Said thresholds can vary over time, such that the determination of the index of susceptibility is variable over time.

The thresholds can in particular vary as a function of absolute time or relative time such as detailed above.

In another embodiment of the invention, which could be combined with the embodiment detailed before, the index of susceptibility to stimulation can be determined at least in part by implementing, by the means of analysis 5, an algorithm for automatic classification of measured data determined from the measured signal S.

Said measured data can be the measured signal S itself or data calculated from the measured signal S such as detailed above, meaning for example an energy from a spectrum of the measured signal S, a frequency of a predefined pattern identified in the measured signal or even a level of muscular activity.

Said automatic classification algorithm is for example defined during a preliminary automatic learning step. Such a preliminary automatic learning step is known from the literature. It may comprise a transfer learning operation with which to change input database, for example for applying an input database, which could be smaller, an algorithm trained on another database, which could be larger (to give a nonlimiting example: apply to people 20 to 25 years old results obtained on people 40 to 45 years old). “Automatic classification algorithm” is understood to mean an algorithm suited for automatically classifying measured data, meaning associating them with a class based on qualitative or quantitative rules characterizing the measured data.

Said class associated with the measured data can be selected from a class database or can be a value interpolated from a class database.

A “class” can thus be for example an identifier, for example an alphanumeric identifier, or even a numeric value, in particular an integer or real value.

The index of susceptibility to stimulation can then be determined based on the resulting class.

The resulting class can directly supply a value of the index of susceptibility to stimulation or can provide intermediate data, in particular intermediate data relating to the measured signal S such as identification of a predefined pattern in the measured signal S, for example identification of a K-complex pattern or “spindle”. The intermediate data are then used to determine an index of susceptibility to stimulation, for example by processing and comparing with thresholds such as detailed above.

Such an algorithm can for example implement a neural network, support vector machine (or large margin separator), decision tree, random forest of decision trees, genetic algorithm or even factor analysis, linear regression, Fisher discriminant analysis, logistic regression or other methods known from the classification field.

Such an algorithm may comprise a plurality of parameters which define qualitative or quantitative rules based on which the automatic classification algorithm automatically classifies the measured data.

Such parameters are for example the weight of certain neurons or all neurons for an algorithm implementing a neural network.

At least one parameter from the automatic classification algorithm and/or a class database can be predefined and recorded in the memory 6 of the device 1 and form operating parameters of the device 1.

As indicated above, said parameter from the automatic classification algorithm and/or class database may vary over time, such that the determination of the index of susceptibility may change over time.

Said parameter from the automatic classification algorithm and/or class database may in particular vary as a function of absolute time or relative time as detailed above.

The parameters for the automatic classification algorithm can for example be predefined during a supervised automatic learning step, or more or less automatically determined, for example by implementing a semi-supervised, partially supervised or unsupervised automatic learning step or by reinforcement. As indicated before, the automatic learning step may comprise a transfer learning operation.

The class database may also be predefined during such a learning step.

Such an automatic learning step may be implemented based on a learning sample of measured data.

Finally, the stimulation step may comprise a substep of emitting an acoustic signal A.

For this purpose, means of emission 4 are designed for emitting an acoustic signal A, audible by the person, and synchronized with the person's predefined brain wave temporal pattern M1 if it is assessed that the person is in a state susceptible to stimulation.

For that purpose, the means of emission 4 comprise for example at least one acoustic transducer 10 and one control electronics 11.

The control electronics 11 is in particular able, in soft real-time, to receive the measured signal S from the means of acquisition 3 and order the acoustic transducer 10 to emit an acoustic signal A synchronized with a predefined temporal pattern T of a slow brain wave of the person P.

“Soft real-time” is understood to mean an implementation of the stimulation operation such that time constraints on this operation, in particular on the length or frequency of repetition of this operation, are respected on average over a predefined total implementation time, for example, of a few hours. It is in particular understood that the implementation of said operation may sometimes exceed said time constraints whereas the average operation of the device 1 and the average implementation of the method complies with them over the predefined total implementation time. Time limits can in particular be defined beyond which the implementation of the stimulation operation must be stopped or paused.

To allow such a soft real-time implementation, a maximum distance between the means of acquisition 3 means of emission 4, means of analysis 5 and memory 6 can be less than about one meter and preferably below a few tens of centimeters. In this way, sufficiently quick communication between the elements of the device 1 can be guaranteed.

The means of acquisition 3, means of emission 4, means of analysis 5 and memory 6 can for example be housed in cavities of the support element 2 clipped onto the support element 2 or even attached to the support element 2 for example by adhering, screwing or any other suitable means of attachment. In an embodiment of the invention, the means of acquisition 3, means of emission 4, means of analysis 5 and memory 6 can be mounted removably on the support element 2.

In an advantageous embodiment of the invention, the control electronics 11 is functionally connected to the means of acquisition 3 and the acoustic transducer 10 via wired connections 10. In this way, the exposure of the person P to electromagnetic radiation is reduced.

The one or more acoustic transducers 10 are capable of emitting an acoustic signal A stimulating at least one inner ear of the person P.

In a first embodiment shown, in particular on FIGS. 1 and 2, an acoustic transducer 10 is an osteophonic device stimulating the inner ear of the person P by bone conduction.

This osteophonic device 10 can for example be suitable for placement near the ear, for example there above as shown in FIG. 1, in particular on an area of skin covering a cranial bone.

In a second embodiment, the acoustic transducer 10 is a speaker stimulating the inner ear of the person P by an auditory canal leading to said inner ear.

The speaker can be arranged outside of the ear of the person P or in the auditory canal.

The acoustic signal A is a modulated signal falling at least partially in a frequency range audible by a person P, for example the range extending from 20 Hz to 30kHz.

The control electronics 11 receives the measured signals S from the means of acquisition 3, which could be preprocessed as detailed above.

If the measured signals S received by the control electronics 11 are not preprocessed, the control electronics 11 can in particular implement one and/or another of pre-processing described above.

The control electronics 11 is next capable of implementing an operation to stimulate brain waves of the person P; the operation is now going to be described in greater detail.

The brain waves can in particular be slow brain waves.

“Slow brain wave” is understood in particular to mean an electrical brain wave of the person P having a frequency below 5 Hz and over 0.3 Hz. Slow brain wave can be understood to mean an electrical brain wave of the person P having a peak-to-peak amplitude included for example between 10 and 200 μV. Beyond the very low frequency waves below 1 Hz, in particular higher frequency delta waves (usually between 1.6 and 4 Hz) are thus also understood to be slow brain waves. Slow brain wave can again be understood as any type of wave having the frequency and amplitude properties indicated above. Thus for example, phase 2 brain waves called “K-Complexes” can be considered as slow brain waves by the invention.

Generally, the invention can be practiced for example during a sleep phase of the person P (such as identified, for example, in the standards of the AASM, “American Academy of Sleep Medicine”), for example a phase of deep sleep of the person P (commonly called stage 3 or stage 4) or during other sleep phases, for example during light sleep of the person (usually called stage 2).

The invention can also be practiced during an arousal, drowsiness or wakening phase of the person P. The brain waves can then be different from slow brain waves.

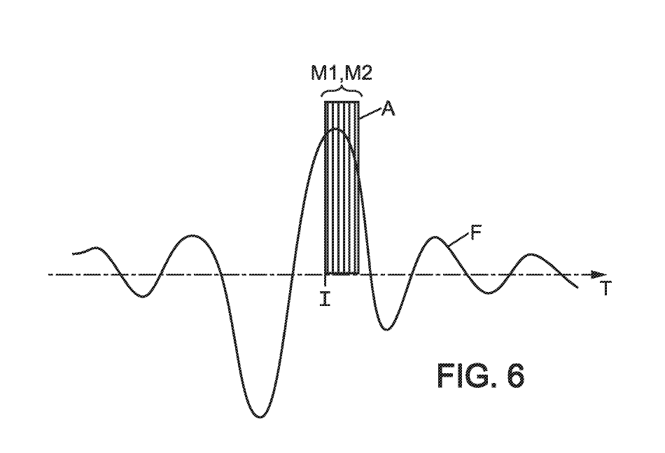

To perform the brain wave stimulation operation, the control electronics 11 is for example capable of determining from the measured signal S a slow brain wave C temporal shape F such as shown in FIG. 6.

In a first embodiment, the temporal shape F is a series of sample points of amplitude values of the measured signal S, which could be preprocessed as indicated above, where said series of measurement points could be interpolated or resampled.

In a second embodiment, the temporal shape F is a series of amplitude values generated by a phase locked loop (PLL).

The phase locked loop is such that the instantaneous phase of the temporal shape F at the output of said loop is synchronized with the instantaneous phase of the measured signal S.

The phase locked loop can be implemented by analog or digital means.

It is therefore understood that the temporal shape F is a representation of the brain wave C which can be obtained directly or can be obtained by a phase locked loop with which to get a cleaner signal. In particular, the instantaneous phase of the temporal shape F and the brain wave C are synchronized in time. In the present description, “brain wave C” is understood as needed to mean the values taken by the temporal shape F.

The control electronics 11 is able to determine from this temporal shape F at least one instant I for synchronization between the predefined temporal pattern M1 of slow brain wave C and the predefined temporal pattern M2 of the acoustic signal A.

Then, the control electronics 11 is able to command the acoustic transducer 10 so that the predefined temporal pattern M2 of the acoustic signal A is emitted at the synchronization instant I.

The predefined temporal pattern M1 of slow brain wave C is therefore a pattern of amplitude values and/or phases of the temporal shape F representing the slow brain wave C. In particular, the predefined temporal pattern M1 can be a succession of phase values of the temporal shape F and can therefore in particular be independent of the absolute value of the amplitude of the temporal shape F.

The predefined temporal pattern M1 can also be a succession of relative values of the amplitude of the temporal form F. Said relative values are for example relative to an amplitude maximum of the predefined or stored temporal shape F.

In an embodiment of the invention, the predefined temporal pattern M1 can thus for example correspond to a local temporal maximum of the slow brain wave C, a local temporal minimum of the slow brain wave C or even a predefined succession of at least one local temporal maximum and at least one local temporal minimum of the slow brain wave C.

The predefined temporal pattern M1 can also correspond to a portion of such a maximum, minimum or such a succession for example of a rising front, descending front or even a plateau.

In the same way, the predefined temporal pattern M2 of the acoustic signal can be a pattern of amplitude values and/or phases of the acoustic signal A.

In a first embodiment, the acoustic signal is for example an intermittent signal as shown in FIG. 6. This intermittent signal is for example emitted during a time less than a period of one slow brain wave. The time of the intermittent signal is for example less than a few seconds, preferably less than one second.

In an example given purely for information and without limitation, the acoustic signal A is for example a pink-noise burst of 1/f type with a time length of 50 to 100 ms with a rise and drop-off time of a few milliseconds. Still without limitation and for making the ideas more concrete, in this example the predefined temporal pattern M1 of the slow brain wave C can for example correspond to a rising front of a local maximum of the slow brain wave C. The predefined temporal pattern M2 of the acoustic signal A can then for example be a rising front of the pink-noise burst. In this example, the instant I for synchronization between the predefined slow brain wave C temporal pattern M1 and the acoustic signal A predefined temporal pattern M2 might for example be defined such that the rising front of the pink noise burst A and the rising front of the local maximum of the slow brain wave C are synchronized, meaning concomitant.

In another embodiment, the acoustic signal A can be a continuous signal. The length of the acoustic signal A can then in particular be longer than one period of the slow brain wave C. “Continuous signal” is understood in particular to mean a signal with a length that is long compared to the slow brain wave C.

In this embodiment, the acoustic signal A can be modulated over time in amplitude, frequency or phase and the predefined temporal pattern M2 of the acoustic signal A can then be such a time modulation.

Alternatively, the continuous acoustic signal A does not have to be time modulated, for example in a way which is now going to be described.

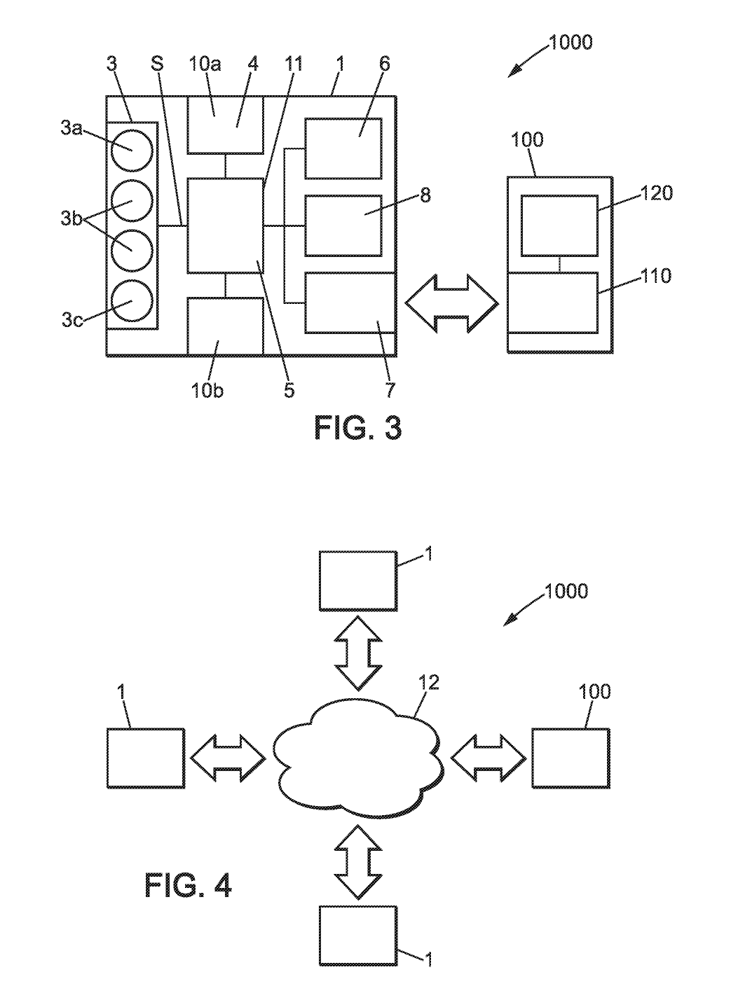

The device 1 can comprise at least two acoustic transducers 10, in particular one first acoustic transducer 10a and one second acoustic transducer 10b as shown in FIG. 3. The first acoustic transducer 10a is capable of emitting an acoustic signal A1 stimulating a right inner ear of the person P. The second acoustic transducer 10b is capable of emitting an acoustic signal A2 stimulating a left inner ear of the person P.

In particular the first and the second acoustic transducer 10a, 10b can then be controlled in a way such that the acoustic signals A1 and A2 are binaural acoustic signals A. For this purpose, the acoustic signals A1 and A2 can be continuous signals with different frequencies.

Such acoustic signals A1, A2 are known for generating intermittent bursts in the brain of the person P, called in particular binaural beats.

Still without limitation and for making the ideas more concrete, in this example the predefined temporal pattern M1 of the slow brain wave C can for example again correspond to a rising front of a local maximum of the slow brain wave C. The predefined temporal patterns M2 of the acoustic signals A1, A2 can additionally be plateaus of acoustic signals A1, A2 corresponding in time to said intermittent bursts generated in the brain of the person P. In this example, the instant R for synchronization between the predefined temporal pattern M1 of slow brain wave C and the predefined temporal patterns M2 of acoustic signals A1, A2 can for example be defined such that an intermittent impulse generated in the brain of the person P is synchronized in time with the rising front of the local maximum of the slow brain wave C.

FIG. 6 shows an example of predefined temporal patterns M1 and M2.

One and/or another among a sound level, length, spectrum and temporal pattern M2 of the acoustic signal A can be predefined and recorded in memory 6 of device 1.

Said one and/or another among a sound level, length, spectrum and temporal pattern M2 of the acoustic signal A can be formed from operating parameters of device 1.

As indicated above, said one and/or another among a sound level, length, spectrum and temporal pattern M2 of the acoustic signal A can vary over time such that emission of the acoustic signal A is variable over time.

Said one and/or another among a sound level, length, spectrum and temporal pattern M2 of the acoustic signal A can in particular vary depending on absolute time or relative time as described above.

The acoustic signal A can thus be emitted as a function of said operating parameters.

Depending on the embodiment and depending on the temporal pattern M1 selected, various embodiments are conceivable for determining the synchronization instant I.

Similarly, one and/or another among a brain wave phase of the person and a predefined brain wave temporal pattern M1 of the person P can be predefined and recorded in memory 6 of device 1.

Said one and/or another among a brain wave phase of the person and a predefined brain wave temporal pattern M1 of the person P can be formed from operating parameters of device 1.

Here again, said one and/or another among a brain wave phase of the person and a predefined brain wave temporal pattern M1 of the person P can vary over time such that synchronized emitting the acoustic signal A is adjusted over time.

Said one and/or another among a brain wave phase of the person and a predefined brain wave temporal pattern M1 of the person P can in particular vary as a function of absolute time or relative time as described above.

The acoustic signal A can thus be emitted so as to be synchronized depending on said operating parameters.

Additionally, for determining the instant I, the control electronics 11 can for example compare the amplitude values of the measured signal S, which could be filtered and/or normalized, with an amplitude threshold.

In the example given above, purely without limitation, the predefined temporal pattern M1 of slow brain wave C corresponds to a rising front of a local maximum of the slow brain wave C. An instant I then corresponds to an instant of exceeding the amplitude threshold, or a predefined time immediately following such an instant of exceeding. The control electronics 11 can in that way command the acoustic transducer 4 so that the predefined temporal pattern M2 of the acoustic signal A is synchronized in time with said instant I.

It is of course understood that the speed of communication between the means of acquisition 3, acoustic transducer 10 and control electronics 11 serves in particular to assure a reliable synchronization and optimal implementation of the stimulation operation.

In an embodiment in which the temporal form F is a series of amplitude values generated by a phase locked loop, it is possible to determine said instant I from said phase locked loop, by threshold detection or by prediction of future values from the temporal form F.

In this embodiment, the temporal form F can in particular be less noisy than the measured signal S and allow easier determination of the instant I of synchronization. In this way it is thus easier to use phase values from the temporal form F for identifying the instant I.

The device 1 can further comprise means of data transmission 7 to the remote server 100. The means of data transmission 7 can be mounted on the support element 2 as described above for the means of acquisition 3, means of emission 4 and means of analysis 5. The means of data transmission 7 can be controlled by electronics of device 1, for example the control electronics 11.

The means of data transmission 7 can advantageously comprise a wireless communication module, for example a module implementing a protocol such as Bluetooth and/or Wi-Fi.

In this way, when the person P is in a sleep period, they are not bothered by cables, in particular if it is necessary to transmit data during the sleep period.

As shown in FIG. 3, the remote server 100 can also comprise means of data transmission 110.

The means of data transmission 7 of the device 1 and the means of data transmission 110 of the remote server 100 are capable of communicating with each other, directly (point-to-point communication) or via a wide area network, for example the Internet.

More specifically, the means of data transmission 7 of the device 1 and the means of data transmission 110 of the remote server 100 are able to exchange data.

Thus, the means of data transmission 7 of the device 1 can in particular be able to transfer measured signals S acquired by the means of acquisition 3 to the means of data transmission 110 of the remote server 100. Such a transfer can in particular be implemented after a sleep period of the person P.

Similarly, the means of data transmission 110 of the remote server 100 can in particular be able to transfer second operating parameters to the means of data transmission 7 of the device 1.

In an embodiment of the invention, the remote server 100 can be capable of communicating with a plurality of devices 1 respectively suitable for being worn by a plurality of people P.

In this embodiment, each device 1 from the plurality of devices 1 can transmit to the remote server 100 operating data comprising at least one measured signal S acquired by means of acquisition 3 of said device 1.

The remote server 100 can thus receive a plurality of operating data respectively associated with a plurality of devices 1.

The operating data received by the remote server 100 from one or more devices 1 are associated with the first operating parameters.

“First operating parameters” is understood to mean operating parameters used by a device for implementing a stimulation step which was used to acquire the measured signal S included in the operating data sent to the remote server 100. The first operating parameters are thus for example operating parameters recorded in a device 1 during production of said device or during a use prior to a personalization method according to the invention.

The remote server 100 can be capable of communicating with the device 1 or with a plurality of devices 1 by means of a wide area network 12, for example the Internet. The device(s) 1 can be directly connected to the wide-area network 12, by the means of data transmission 7 thereof, or else be connected to said wide-area network 12 by means of a mediation device, for example a base station, computer or smart phone.

The remote server 100 also comprises means of processing 120 capable of processing operating data for determining second operating parameters.

“Second operating parameters” means operating parameters determined by the means of processing based on operating data. The second operating parameters can in particular be identical to the first operating parameters or different from the first operating parameters.

The means of processing 120 can for example comprise one or more processors and also one or more appropriate memories.

The means of processing 120 are able and intended to implement a step of processing operating data received from the device 1 during which second operating parameters are determined from an analysis of the operating data.

In an embodiment in which the remote server 100 receives a plurality of operating data associated with a plurality of devices 1, the processing step can in particular include the analysis of said plurality of operating data so as to determine second operating parameters to send and store in the memory of at least one device among the plurality of devices 1.

In a first embodiment, the step of processing operating data can comprise the analysis of the measured signal S by the processing means 120 for identifying at least one parasitic frequency of the measured signal S.

To do that, the processing means 120 can in particular calculate a harmonic spectrum of the measured signal S and compare the amplitudes of one or more frequencies of said spectrum with average values of energy or spectral amplitude, or with thresholds of maximum energy or spectral amplitude, so as to detect spectral amplitudes that are too large.

Said average values of energy or spectral amplitude or thresholds of maximum energy or spectral amplitude can in particular be determined from a plurality of devices 1 for example from a plurality of operating data associated with a plurality of devices 1 as detailed above.

When at least one parasitic frequency has been identified in the measured signal S, it is then possible to determine second operating parameters depending on said parasitic frequency.

The second operating parameters are next sent from the remote server 100 and stored in the memory 6 of the device 1 during a step of transmission and storage.

In the second embodiment, which can in particular be combined with the first embodiment described above, the step of processing operating data can include searching the measured signal S for at least one temporal or frequency pattern indicating wakening or beginning of wakening of the person P, said temporal pattern being next in time after emitting an acoustic signal A.

“Said temporal pattern being next in time after emitting an acoustic signal” is understood to mean that the temporal pattern sought was acquired by the means of acquisition 3 after emitting an acoustic signal A, in a certain time range which can follow immediately after emitting the acoustic signal A or be delayed for considering biological reaction time of the person P to the acoustic signal A.

To do that, the means of processing 120 can implement a frequency analysis by Fourier transform of at least one portion of the measured signal S following emitting an acoustic signal A, followed as necessary by the implementation of a deep sleep detection algorithm. In this way, it is possible to detect whether emission of the acoustic signal generated wakening or beginning of wakening of the person P.

From this analysis, the means of processing 120 can determine an indicator of wakening under the effect of the stimulation.

If said indicator of wakening indicates that emitting the acoustic signal generated a wakening or beginning of wakening of the person P, the second operating parameters can be determined so as to prevent a future occurrence of the situation.

To do that, the processing means 120 can for example determine second operating parameters comprising more specifically operating parameters used by the means of analysis 5 detailed above.

The second operating parameters can thus comprise one and/or another among at least one predefined threshold for energy from a spectrum of the measured signal S, at least one predefined threshold for time frequency of a predefined pattern identified in the measured signal, or even at least one predefined threshold of muscular activity level such as detailed above.

Preferably, said thresholds from the second operating parameters are below the thresholds from the first operating parameters of the device 1 so as to prevent wakening or beginning of wakening of the person P during later stimulations.

The second operating parameters are next sent from the remote server 100 and stored in the memory 6 of the device 1 during a step of transmission and storage.

In a third embodiment, which can in particular be combined with the first and second embodiment described above, the step of processing operating data can comprise the comparison of a portion of the measured signal acquired after emitting an acoustic signal A with a baseline portion of the measured signal, so as to determine an impact indicator for the stimulation.

Here again, “a portion of the measured signal acquired after emitting an acoustic signal A” is understood to mean that said portion of the measured signal was acquired by the means of acquisition 3 after emitting an acoustic signal A, in a certain time range which can follow immediately after emitting the acoustic signal A or be delayed for considering biological reaction time of the person P to the acoustic signal A.

Said time range can for example extend over several seconds after emitting the acoustic signal A.

“A baseline portion of the measured signal” is understood to mean a portion of the signal measured before emitting any acoustic signal A, or again a portion of the signal measured sufficiently far in time from emitting any acoustic signal A so that the person P is no longer considered as influenced by emission of an acoustic signal A. Such a baseline portion can be an average made over several portions of measured signal, in particular several portions of the measured signal preceding each emission of an acoustic signal A.

The means of processing 120 can for example determine a difference between the averages of the portion of the measured signal acquired after emitting an acoustic signal A and the reference portion of the measured signal, and a stimulation impact indicator can be determined from said difference.

Additionally, as detailed above, the processing means 120 can implement a frequency analysis by Fourier transformation of the portion of the measured signal S after emitting an acoustic signal A so as to detect whether emitting the acoustic signal generated wakening or beginning of wakening of the person P and determine an indicator of wakening under the effect of the stimulation.

A composite stimulation effect indicator can then be determined from the stimulation impact indicator and the indicator of wakening under the effect of stimulation.

The second operating parameters can then be determined from the composite stimulation effect indicator.

To do that, the processing means 120 can for example determine second operating parameters comprising more specifically operating parameters used by the means of emission 4 detailed above.

The second operating parameters can thus comprise one and/or the other among at least the sound level, length, spectrum or temporal pattern M2 of the acoustic signal A, or even at least one among a brain wave phase of the person and a predefined brain wave temporal pattern M1 such as detailed above.

The second operating parameters are next sent from the remote server 100 and stored in the memory 6 of the device 1 during a step of transmission and storage.

In an embodiment of the invention, the second operating parameters are determined by implementing an automatic classification algorithm on the operating data by means of processing 120.

Said automatic classification algorithm is for example defined during a preliminary automatic learning step. As indicated before, the automatic learning step may comprise a transfer learning operation.

“Automatic classification algorithm” is understood to mean an algorithm suited for automatically classifying operating data sent by the device 1, meaning associating them with a class based on qualitative or quantitative rules characterizing the operating data. “Automatic classification algorithm” is also understood to mean broadly regression algorithms able to associate a class which is a real value with operating data sent by the device 1.

Said class associated with the operating data can be selected from a class database or can be a value interpolated from a class database.

A “class” can thus be for example an identifier, for example an alphanumeric identifier, or even a numeric value, in particular an integer or real value. In the case where the class is a real value, one then speaks of a regression algorithm.

Said algorithm can be implemented directly on the operating data themselves or the operating data can be preprocessed by filtering or other means before implementing the operating algorithm.

Such an algorithm can for example implement a neural network, support vector machine (or large margin separator), decision tree, random forest of decision trees, genetic algorithm or even factor analysis, linear regression, Fisher discriminant analysis, logistic regression or other methods known from the classification field.

Such an algorithm may comprise a plurality of parameters which define qualitative or quantitative rules based on which the automatic classification algorithm automatically classifies the measured data.

Such parameters are for example the weight of certain neurons, of all neurons, or even of connections between the neurons for an algorithm implementing a neural network.

The parameters for the automatic classification algorithm can for example be predefined during a supervised automatic learning step, or more or less automatically determined, for example by implementing a semi-supervised, partially supervised or unsupervised automatic learning step or by reinforcement. As indicated before, the automatic learning step may comprise a transfer learning operation.

The class database may also be predefined during such a learning step.

Such an automatic learning step may be implemented based on a learning sample of measured data.

The second operating parameters can then be determined from the resulting class.

Said class can directly provide a value for second operating parameters or can be used for determining one or more values of second operating parameters, for example by comparison with a database of second operating parameters and/or by interpolation between predefined values of second operating parameters in such a database.

Thus, the resulting class can serve to select a user behavior under the effect of the stimulation among several user behaviors under the effect of the stimulation recorded in a database and associated in said database with predefined second operating parameters.

To do that, the processing means 120 can for example determine second operating parameters comprising more specifically operating parameters used by the means of emission 4 detailed above.

The second operating parameters can thus comprise one and/or the other among at least the sound level, length, spectrum or temporal pattern M2 of the acoustic signal A, and/or even at least one among a brain wave phase of the person and a predefined brain wave temporal pattern M1, and/or at least one parameter from an automatic classification algorithm and/or a database of classes from an automatic classification algorithm implemented by analysis means 5 of the device 1 such as detailed above.

In an embodiment of the invention in which the second operating parameters comprise at least one parameter from an automatic classification algorithm and/or a database of classes from an automatic classification algorithm implemented by means of analysis 5 from a device 1, the remote server 100 can implement an algorithm such as indicated above.

The remote server 100 can first implement an automatic classification algorithm associated with, in particular similar to, said automatic classification algorithm implemented by means of analysis 5 for the device 1.

However, the automatic classification algorithm implemented by the remote server 100 can be applied to much more extensive input data than the measured data to which the associated automatic classification algorithm implemented by the device 1 is applied.

In fact, the associated automatic classification algorithm implemented by the device 1 operates in real time, meaning that at a given instant it only has access to measured data recorded prior to said instant. On the other hand, the automatic classification algorithm implemented by the remote server 100 operates off-line and the input data to which said algorithm is applied can thus comprise, for each instant, measured data recorded by a device 1 before and after said instant.

Additionally, the remote server 100 can receive measured signals S acquired by a device over several operating period of the device, for example during several sleep periods of the person P. The input data to which the algorithm implemented by the remote server 100 is applied can thus comprise, for each instant, measured data recorded during different operating periods of the device 1.

It is therefore understood that the algorithm implemented by the remote server 100, however similar to the classification algorithm implemented by the means of analysis 5 of the device 1, can serve to obtain more precise results.

The algorithm implemented by the remote server 100 can thus be used for labeling the measured data received from the device 1, meaning determine the expected output values from the classification algorithm implemented on the device 1 for the various measured data input.

After labeling the measured data, the remote server 100 can determine an updated classification algorithm for the device 1.

To do that, the remote server 100 can implement an automatic learning operation for the automatic classification algorithm from the device 1, in particular learning by reinforcement, from labeled measured data. The automatic learning operation can be implemented based on an initial state built up by the automatic classification algorithm currently implemented on the device 1.

The second operating parameters can next be determined from the updated classification algorithm.

The second operating parameters are next sent from the remote server 100 and stored in the memory 6 of the device 1 during a step of transmission and storage.

Once the second operating parameters are sent from the remote server 100 and stored in the memory 6 of the device 1 during the transmission and storage step, the device 1 can then implement a second acoustic brain wave stimulation step in which at least one of the acquisition a), analysis b) and emission c) substeps is done depending on the second operating parameters.

Here “second brain wave stimulation step” is understood to mean a step of stimulation implemented after sending second operating parameters from the remote server. It is in particular understood that when a plurality of stimulation steps were implemented before the operating data processing step (on the remote server), said “second brain wave stimulation step” might not be the second stimulation step implemented but a later implementation.

1. A method for personalized acoustic brain wave stimulation of a person comprising:
A first step of acoustic brain wave stimulation of a person implemented by a device for acoustic brain wave stimulation adapted to be worn by the person, wherein the stimulation device comprises memory storing first operating parameters, and the initial stimulation step comprises the substeps of:
a) Acquisition of at least one measured signal representative of a physiological signal from the person;
b) Analysis of the measured signal in order to assess whether the person is in a state susceptible to stimulation; and
c) if the assessment shows that the person is in a state susceptible to stimulation, emitting an acoustic signal, audible by the person, and synchronized with a predefined brain wave temporal pattern of the person,
wherein at least one of the substeps of acquisition a), analysis b), and emission c) is done depending on the first operating parameters;
A step of sending operating data from the device to a remote server, wherein said operating data comprise said measured signal;
A step of operating data processing, on the remote server, for determining second operating parameters;
A step of transmission and storage of the second operating parameters from the remote server into the memory of the device;
A second step of acoustic brain wave stimulation implemented by the device wherein the memory of the device stores the second operating parameters, and wherein at least one of the substeps of acquisition a), analysis b), and emission c) is done depending on the second operating parameters.

2. The method according to claim 1, wherein the operating parameters comprise at least one parasitic frequency of the measured signal,
and wherein the substep of acquisition a) comprises a frequency filtering of said parasitic frequency in the measured signal.

3. The method according to claim 1, wherein the operating parameters comprise at least one energy threshold for a spectrum of the measured signal,
and wherein the substep of analysis b) comprises the comparison of an energy from spectrum of the measured signal with said threshold.

4. The method according to claim 1, wherein the operating parameters comprise at least one temporal frequency threshold for a predefined pattern from the measured signal,
and wherein the substep of analysis b) comprises the identification of said predefined pattern in the measured signal and the comparison of a frequency of said pattern in the measured signal with said threshold.

5. The method according to claim 1, wherein the operating parameters comprise at least one muscular activity level threshold,
and wherein the substep of analysis b) comprises the determination of a muscular activity level from the measured signal and the comparison of said muscular activity level with said threshold.

6. The method according to claim 1, wherein the substep of analysis b) is implemented at least in part by an algorithm for automatic classification of measured data determined,from the measured signal,
and wherein the operating parameters comprise at least one parameter from said automatic classification algorithm and/or one database of classes from said automatic classification algorithm.

7. The method according to claim 1, wherein the operating parameters comprise at least one parameter selected from a list including sound level, length, spectrum and time pattern of an acoustic signal,
and wherein the acoustic signal emitted during the substep of emission c) is emitted as a function of said parameter.

8. The method according to claim 1, wherein the operating parameters comprise at least one parameter selected from a list including a person's brain wave phase and a predefined brain wave temporal pattern of the person,
And wherein the acoustic signal emitted during, the substep of emission c) is emitted so as to be synchronized as a function of said parameter.

9. The method according to claim 1, wherein the processing step comprises analyzing the measured signal on the remote server for identifying at least one parasitic frequency of the measured signal,
and wherein the second operating parameters are determined depending on said parasitic frequency.

10. The method according to claim 1, wherein the processing step comprises searching the measured signal for at least one predefined pattern indicating wakening or beginning of wakening of the person and following emitting an acoustic signal, so as to determine an indicator of wakening under the effect of the stimulation,
and wherein the second operating parameters are determined depending on said wakening indicator.

11. The method according to claim 1, wherein the processing step comprises comparing a portion of the measured signal acquired after emitting an acoustic signal with a baseline portion of the measured signal, so as to determine a stimulation impact indicator,
And wherein the second operating parameters are determined depending on said impact indicator.

12. The process according to claim 10, wherein determination of the second operating parameters depending on the stimulation impact indicator includes implementing an automatic classification algorithm, wherein said automatic classification algorithm is preferably defined during a preliminary automatic learning step.

13. The method according to claim 1, wherein the first step of acoustic brain wave stimulation of a person is implemented a plurality of times by a plurality of respective devices, respectively adapted to be worn by a plurality of respective people,
wherein the step of transmitting operating data to the remote server is implemented a plurality of times from said plurality of respective devices, so as to respectively send a plurality of respective operating data, respectively comprising at least one measured signal from each of said respective devices,
and wherein the processing step comprises the analysis of said plurality of operating data so as to determine second operating parameters to be sent and stored in the memory of at least one device among the plurality of devices.

14. The method according to claim 1, wherein the first stimulation step is repeated a plurality of times by a device during a time of operating said device,
and wherein the step of transmitting operating data to a remote server from the device is implemented after said operating time, wherein the operating data comprise at least one measured signal for each of the repetitions of the first stimulation step.

15. The process according to claim 14, wherein the operating time of the device extends over several hours, preferably at least eight hours.

16. The method according to claim 1, wherein emitting an acoustic signal synchronized with a predefined brain wave temporal pattern comprises:
Determining a brain wave temporal shape from the measured signal,
Determining from said brain wave temporal shape at least one instant for synchronization of the predefined brain wave temporal pattern with a predefined acoustic signal temporal pattern, and
Commanding an acoustic transducer of the device so that the predefined acoustic signal temporal pattern is emitted at said synchronization instant.

17. The method according to claim 1, wherein the brain wave is a slow brain wave having a frequency below 5 Hz and over 0.3 Hz.

18. The method according to claim 1, wherein the acoustic signal is an intermittent signal and wherein an acoustic signal length is less than a brain wave period, preferably less than a few seconds, preferably less than one second.

19. The method according to claim 1, wherein the acoustic signal is a continuous signal and an acoustic signal length is greater than a brain wave period.

20. The method according to claim 1, wherein the predefined brain wave temporal pattern corresponds to a brain wave local temporal maximum, a brain wave local temporal minimum, a rising or descending front of a brain wave local maximum or minimum, a predefined succession or at least one brain wave local temporal maximum or minimum, or a rising or descending front of such a succession.

21. A personalized acoustic brain wave stimulation system for a person, wherein the system comprises an acoustic brain wave stimulation device for a person and a remote server,
wherein the device comprises:
A memory adapted to store operating parameters comprising at least one among first operating parameters and second operating parameters,
an acquisition element of at least one measured signal representative of a physiological signal from the person,
an analysis element of the measured signal in order to assess whether the person is in a state susceptible to stimulation, and
of an emission element designed for emitting an acoustic signal, audible by the person, and synchronized with the person's predefined brain wave temporal pattern if it is assessed that the person is in a state susceptible to stimulation,
wherein the at least one among the acquisition element, analysis element and of emission element is adapted to operate depending on operating parameters stored in memory,
wherein the device and the remote server comprise respective elements of data transmission designed for:
transmitting operating data from the device to the remote server, wherein said operating data comprises at least said measured signal and
transmitting second operating parameters from the remote server to the device and storing said second operating parameters in the memory of the device;
and wherein the remote server comprises an element of processing operating data in order to determine second operating parameters.
